(19) United States
(12) Patent Application Publication
(10) Pub. No.: US 2024/0182535 A1
(43) Pub. Date: Jun. 6, 2024

(54) GRANULOCYTE MACROPHAGE-COLONY STIMULATING FACTOR MUTANTS

(71) Applicant: Partner Therapeutics, Inc., Lexington, MA (US)

(72) Inventors: Michael FELDHAUS, Lexington, MA (US); Jeffrey YOST, Lexington, MA (US)

(21) Appl. No.: 18/033,252

(22) PCT Filed: Oct. 25, 2021
(86) PCT No.: PCT/US21/56413

§ 371 Date: Apr. 21, 2023

Related U.S. Application Data
(60) Provisional application No. 63/177,481, filed on Apr. 21, 2021.
(60) Provisional application No. 63/105,425, filed on Oct. 26, 2020.

(51) Int. Cl. C07K 14/535; A61K 35/16; A61K 38/19

(52) U.S. Cl. CPC C07K 14/535; A61K 35/16; A61K 38/193

(57) ABSTRACT

The present invention is based on the discovery that amino acid substitutions in the sequence of sargramostim yield a product with simplified manufacturability.

### CROSS-REFERENCE TO RELATED APPLICATIONS

This application claims the benefit of and priority to U.S. Provisional Patent Application Nos. 63/105,425, filed Oct. 26, 2020 and 63/177,481, filed Apr. 21, 2021, the content of which are hereby incorporated by reference in their entirety.

### FIELD

The present invention relates generally to compositions and methods involving to mutant granulocyte-macrophage colony-stimulating factor (GM-CSF).

### SEQUENCE LISTING

The instant application contains a Sequence Listing that has been submitted in ASCII format via EFS-Web and is hereby incorporated by reference in its entirety. Said ASCII copy, created on Oct. 20, 2021, is named “PNR-003PC_ST25.txt” and is 4,096 bytes in size.

### BACKGROUND

Colony Stimulating Factor, CSF, refers to a family of four glycoproteins that control and coordinate cell production by widely scattered deposits of marrow cells. These include: Granulocyte-Macrophage CSF (GM-CSF), Granulocyte colony CSF (G-CSF), Macrophage colony CSF (M-CSF) and multipotential colony-stimulating factor (IL-3). These lymphokines can induce progenitor cells found in the bone marrow to differentiate into specific types of mature blood cells. The particular type of mature blood cell that results from a progenitor cell depends upon the type of CSF present. See Metcalf D. Cancer Immunol Res. 2013, 1(6): 351-356.

GM-CSF is a hematological growth factor that regulates the production, migration, proliferation, differentiation and function of hematopoietic cells. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it modulates in vitro proliferation, differentiation, and survival of hematopoietic progenitor cells, but is present in circulating blood in only picomolar concentrations (10⁻¹⁰ to 10⁻¹² M). See Alexander W S. Int Rev Immunol. 1998, 16:651-682; Gasson J C. Blood. 1991, 77:1131-1145; Shannon M F et al. Crit Rev Immunol. 1997, 17:301-323, Barreda D R et al. Dev Comp Immunol. 2004, 28:509-554 and Metcalf D. Immunol Cell Biology. 1987, 65:35-43.

Human GM-CSF (hGM-CSF) is synthesized as a 144 amino acid residue precursor protein with a 17 amino acid signal peptide. This precursor protein is processed to yield a 127 amino acid mature protein with a predicted molecular mass of 14.4 kDa. It has two disulfide linkages that migrates as a broad band of 15-30 kDa due to glycosylation and sialylation. N-linked glycans are located at Asn²⁷ and Asn³⁷ and multiple potential sites for O-linked glycosylation exist at Ser5, Ser7, Ser9 and Ser10 but the extent of glycan structures at these sites have not been unambiguously determined. The glycosylation patterns of GM-CSF have been observed to influence its activity, receptor binding, immunogenicity, and half-life. See Lee F. et al. Proc Natl Acad Sci USA Biochem. 1985. 82: 360-4364; Miyatake S. et al. EMBO J. 1985. 4: 2561-2568. Cebon J et al. J Biol. Chem. 1991. 265, 4483-4491; Zhang Q et al. Proc. Natl. Acad. Sci. 2014, 2885-2890.

Recombinant human granulocyte-macrophage colony-stimulating factor (rhu GM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15(1): 19-29; Armitage J O. Blood 1998, 92:4491-4508; Kovacic J C et al. J Mol Cell Cardiol. 2007, 42:19-33; Jacobs P P et al. Microbial Cell Factories 2010, 9:93.

Although there are five classes of heterologous protein production platforms, including bacteria, yeasts, plants, insect cells, and mammalian cells, more than 50% of currently marketed biopharmaceuticals are produced in mammalian cell lines. This is in part due to the inability of the remaining four classes to modify glycoproteins with human-like oligosaccharides. This is of importance as protein-bound glycans influence circulation half-life, tissue distribution, biological activity and immunogenicity.

The GM-CSF expression system influences the pharmacokinetics properties, biological activity and clinical toxicity of GM-CSF. In the clinic, GM-CSF has been produced in Chinese hamster ovary cells (CHO-GM, regramostim), Escherichia coli (E. coli-GM, molgramostim), or yeast (Yeast-GM, sargramostim). See Dorr R T. Clin Therapeutics. 1993. 15(1): 19-29; Walsh G. Nat Biotechnol. 2006, 24:769-776; Jacobs P P et al. Nat Protoc. 2009, 4:58-70; Jacobs P P et al. Microbial Cell Factories 2010, 9:93; Walsh G. Nat Biotechnol. 2018, 36(12): 1136-1145. During the manufacture of GM-CSF, four major GM-CSF isoforms/species are present: “hyper-glycosylated” (˜50 kDa), N- and N-+O-glycosylated (˜20 kDa), O-glycosylated (˜16 kDa) and the non-glycosylated (˜15 kDa) species. Glycosylated GM-CSF is obtained exclusively via recombinant technologies using yeast (Sargramostim) or Chinese hamster ovary (CHO) cell (Regramostim) technologies, which yield complex mixtures of glycoforms, but not using E. coli. The glycan heterogeneity reflects a lack of specificity in CHO-cell posttranslational glycosylation. See Zhang Q et al. PNAS. 111(8): 2885-2890.

The expression vectors for sargramostim contains leader sequences which direct transcription in the rough endoplasmic reticulum and secretion by cells of mature GM-CSF. N-glycosylation occurs when glycans are added to the nitrogen of asparagine (Asn) or Arginine (Arg) side chains, whereas O-glycosylation occurs when glycans are added to the hydroxyl oxygen side chains of serine (Ser) or threonine (Thr) or tyrosine (Tyr) amino acids. Asparagine residues typically require a consensus sequence of Asn-Xxx-Ser/Thr/Cys, where Xxx can be any amino acid except proline, to be N-glycosylated. The rhu GM-CSF protein sequences for sargramostim contain two consensus sequences, Asn-Leu-Ser (NLS) and Asn-Glu-Thr (NET), where the protein can potentially be glycosylated. During manufacture of sargramostim both N-glycosylation sites, Asn²⁷ and Asn³⁷, are glycosylated in some GM-CSF species present in the fermenter. The Thr³⁹ (or Ser³⁹ or Cys³⁹) residue is an important anchor to enable glycosylation at Asn²⁷ or Asn³⁷. Species with N-oligosaccharides attached at asparagine residue 37 are referred to as “hyper-glycosylated” species (30-100 kDa) because these glycoforms contain up to several hundreds of mannose residues. Only hypermannosylation occurs at N37. Prior reports have explored mutagenesis that may impact glycosylation (e.g. Gene 55: 287-293 (1987) and Hehring Inst. Mitt. 83: 1-7 (1988)), however, these studies focused on protein yield and did not assess activity or attempt to streamline purification processes (see Table 2 of Hehring Inst. Mitt. 83: 1-7 (1988), for instance).

During the upstream manufacturing process, four major GM-CSF species, “hyper-glycosylated” (˜50 kDa,), N- and N-+O-glycosylated (˜20 kDa, peak 2), O-glycosylated (˜16 kDa, peak 3) and the non-glycosylated (˜15 kDa, peak 4) species present in partially purified fermenter broth. The “hyperglycosylated” GM-CSF species is considered non-product and is removed in the downstream process resulting in a product, the Bulk Drug Substance (BDS), that contains the three major glycoforms (peaks 2-4). A small amount of oxidized GM-CSF is detected in BDS and DP (≤4%) which is labelled “peak 1” in the T-0075 reversed phase glycoform assay. See Walter P, et al. JCB. 1981. 91 (2 Pt1): 545-50; Cantrell M A, et al. PNAS. 1985. 82:6250-6254. Most proteins synthesized in the rough endoplasmic reticulum undergo glycosylation which is an enzyme-directed site-specific process. See Medzihradszky K F. Methods Mol Biol. 2008. 446:293-316. Varki A et al. Essentials of Glycobiology. Chapter 56. 3^{rd} edition.

In addition to the three major rhu GM-CSF glycoforms currently present in the formulations, there is a “hyper-glycosylated” form that makes up less than 6% of the product. This “hyper-glycosylated” form however is not present in the current sargramostim (LEUKINE) drug product. The isolated “hyper-glycosylated” species was estimated to contain two N-acetylglucosamine residues plus on average of 43 mannose residues which is at the low end of the range known to be present in fermentation broth. Species with N-oligosaccharides attached at asparagine residue 37 are referred to as “hyper-glycosylated” species (30-100 kDa) because these glycoforms contain up to several hundreds of mannose residues. See Strenler K E, et al. 1994. This “hyper-glycosylated” species is entirely removed in the downstream purification processes and none can be detected in the final sargramostim Bulk Drug Substance (BDS) used to manufacture sargramostim.

The final product of sargramostim consists of N-glycosylated and O-glycosylated glycoforms along with non-glycosylated glycoforms. Thus, sargramostim has heterogeneity in its glycoform profile which has been very consistent throughout its licensed history. These three major glycoforms in sargramostim which can be separated on SDS-PAGE gel. The historic average relative amounts of these three glycoforms are reasonably consistent at 28.68%+0.86%, 22.58%+0.73% and 48.74%+0.8%, respectively. Nearly half of the GM-CSF protein in sargramostim is non-glycosylated and slightly less than a third is fully N-glycosylated. Most if not all of the N-glycosylated species are also O-glycosylated. The highly branched N-linked oligosaccharide structure overlays a significant portion of the molecule including the H1 and H5 alpha-helices without obstructing the receptor binding site as well as some of the H6 alpha-helix and C-terminal random coil.

Although the carbohydrate component of recombinant GM-CSF differs based on the cellular source, glycosylation is not required for receptor binding or for in vitro and in vivo biological activity. Yeast rhu GM-CSF and non-glycosylated E. coli-derived rhu GM-CSF have comparable specific activities measured by both receptor affinity and cell proliferation specific activity. See Urdal and Park, 1988; Metcalf D. Cancer 1990: 65:2185-2195.

Endogenous proteins in humans are degraded in vivo at different rates which may vary widely depending on their functions. For instance, hemoglobin lasts for the entire life-time of an erythrocyte and histones have a half-life measured in years while other proteins like ornithine decarboxylase (11 min half-life) have very rapid degradation rates. See Thomas E Creighton (1993). “Chapter 10—Degradation”. Proteins: Structures and Molecular Properties (2nd ed.). WH Freeman and Company. pp. 463-473. ISBN 0-7167-2317-4. In eukaryotes, most short-lived proteins in the intracellular space are degraded by the ubiquitin-proteasome pathway (UPP). See Kybuczkova L, et al. J Cell Mol Med. 2014. 18: 947-961. The UPP plays a central role in cellular homeostasis in the processing and degradation of proteins, including those that regulate cell-cycle progression, apoptosis, and DNA repair. See Heinemeyer W, et al. J Biol Chem. 1997. 272: 25200-25209. Lysosomes also remove unwanted proteins in the cytoplasm and from outside the cell. Material from outside the cell is taken-up through endocytosis, while material from the inside of the cell is digested through autophage. See Settembre C, et al. Nature Reviews Molecular Cell Biology. 2013.14 (5): 283-96. GM-CSF has been shown to have a half-life that can extend from 50 to 85 min. See Cebon et al. Blood. 1988. 72: 1340-1347; Stagg et al 2004. The in vivo biological activity of naturally occurring and recombinant GM-CSF is largely dependent upon their bioavailability. See Dorr R T. Clinical Therapeutics/vol. 15, NO. 1, 1993.

The bioavailability of therapeutic proteins (biotherapeutic or biologic) in humans can also be influenced by the route of administration of the drug. See Dorr R T, Clinical Therapeutics/vol. 15, NO. 1, 1993. Immediately following intravenous (IV) administration nearly all of the therapeutic protein is available, but then may be degraded over time by proteases present in blood plasma. In some cases, SubCutaneous (SC) administration has provided prolonged exposure to proteins with short elimination half-lives by maintaining high plasma concentrations for longer periods and can be better tolerated compared to IV administration. Thus, absorption of a biotherapeutic after SC administration may be influenced by its fate in the extra-cellular matrix (ECM) including the possibility of incomplete bioavailability. See Hale G, et al. Blood. 2004.104(4): 948-955.

Transport of the different isoforms present in sargramostim into the systemic circulation may favor different routes because of their different molecular sizes. The two non-glycosylated rhu GM-CSF Leu23 species in sargramostim (˜14 kDa on SDS PAGE gels, MW=14,430.47 and 14266.31 determined from the amino acid sequence for the full length molecule and for the truncated Ala3 des-A1P2 species), clearly should favor diffusion into blood capillaries and the bioavailability of these protein molecules in the blood stream should, therefore, be quite rapid. The roughly eight O-glycosylated glycoforms (˜16 kDa on SDS PAGE gels, MWs between 14,500 and 16,000 determined by mass spectrometry) containing 1-8 mannose sugar residues might be delivered to systemic circulation through both lymphatic and blood capillaries. The N- and N-+O-glycosylated glycoforms (˜21 kDa on SDS PAGE gels, MW between 16,200 and 19,000 determined by mass spectrometry) containing two n-acetylglucosamine residues and between 6-27 mannose residues, however, given their molecular size might strongly favor diffusion and absorption by the lymphatic pathway.

The GM-CSF expression system can influence the pharmacokinetic parameters, with the extent of glycosylation affecting GM-CSF half-life, distribution and elimination. A relationship between the extent of GM-CSF glycosylation and its distribution, clearance, and activity has been reported using a rat model. The specific activity of GM-CSF measured in vitro was found to be significantly decreased in the largest, most fully glycosylated forms of the protein relative to the smaller, less heavily glycosylated molecules. The effective half-life of GM-CSF in the bloodstream of a rat following a single intravenous bolus injection was shown to be significantly increased by the addition of N-linked carbohydrate. The clearance of GM-CSF in the rat follows biphasic kinetics, and it is the first or c˜phase that is prolonged by the carbohydrate modification. See Donohue R E, et al. Cold Spring Harbor Labs. 1986. 51: 685-692 and Dorr R T Clinical Therapeutics. 1993. 15(1): 19-29.

The “hyper-glycosylated” rhu GM-CSF species has been shown to have a lower biological activity that was statistically significant while the specific activities (SAs) for the 19.5, 16.8 and 15.5 kDa species are quite similar. The SA for the other three glycoforms, N- and N-+O-glycosylated (21 kDa, peak 2), O-glycosylated (16 kDa, peak 3) and non-glycosylated species (14 kDa, peak 4), have been shown not to be statistically different at the 99% confidence level from the values obtained for the parent BDS, sargramostim.

There remains a need for GM-CSF molecules that are amenable to simple purification methods and a largely devoid of “hyper-glycosylated” species.

### SUMMARY

Accordingly, the present invention relates to mutant forms of GM-CSF, which, inter alia, provide a unique glycoform profile, relative to wild type or sargramostim.

In aspects, there is provided a recombinant human GM-CSF protein, comprising an amino acid sequence having at least about 97% identity with SEQ ID NO: 1 or SEQ ID NO: 2 and having a substitution or deletion at position N37, E38 and/or T39 or a position corresponding thereto, e.g. the amino acid at position N37 or a position corresponding thereto may be substituted to a polar and neutral of charge hydrophilic amino acid, such as glutamine (Q), serine (S), threonine (T), proline (P), and cysteine (C); the amino acid at position E38 or a position corresponding thereto may be substituted to a hydrophobic, aliphatic amino acid, such as alanine (A), leucine (L), isoleucine (I), methionine (M), and valine (V). and/or is not a proline; and/or the amino acid at position T39 or a position corresponding thereto may be substituted to an alanine (A), glycine (G), leucine (L), isoleucine (I), methionine (M), and valine (V).

In embodiments, the present recombinant human GM-CSF is functionally similar to sargramostim. In embodiments, the present recombinant human GM-CSF comprises a plurality of molecular forms, e.g. non-glycosylated, O-glycosylated, N-glycosylated and/or N+O glycosylated forms. In embodiments, the present recombinant human GM-CSF is substantially free of hyperglycosylated, e.g. hypermannosylated forms. In embodiments, the present recombinant human GM-CSF resolves as three peaks when quantified by reversed phase high-performance liquid chromatography (RP-HPLC). In embodiments, the present recombinant human GM-CSF provides no substantial peaks at a retention time of less than about 20 minutes when quantified by reversed phase high-performance liquid chromatography (RP-HPLC). In embodiments, the present recombinant human GM-CSF is substantially free of hyperglycosylated, e.g. hypermannosylated forms when purified without the use of organic solvents (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak.

In embodiments, the recombinant human GM-CSF produced in a mammalian cell is functionally enhanced as compared to a recombinant human GM-CSF produced in a yeast cell.

In embodiments, production titers of the recombinant human GM-CSF produced in a mammalian cell is increased as compared to production in a yeast cell.

In embodiments, there is also provided nucleic acid molecules encoding the present recombinant human GM-CSF (e.g. a codon-optimized sequence). In embodiments, there is also provided a non-human host cell expressing the nucleic acid molecule encoding the present recombinant human GM-CSF (e.g. a yeast cell, e.g. a non-methylotrophic yeast cell, e.g. a Saccharomyces cerevisiae. In embodiments, there is also provided a pharmaceutical composition comprising the present recombinant human GM-CSF and a pharmaceutically acceptable excipient or carrier.

In aspects, there is provided a method of treating a patient or subject who is undertaking or has undertaken a cancer therapy, or who is undertaking or has undertaken a bone marrow transplant, and/or who had been acutely exposed to myelosuppressive doses of radiation; the method comprising administering to the patient a therapeutically effective amount of the pharmaceutical compositions described herein.

In aspects, there is provided a method of treating a viral infection, e.g. without limitation an infection with a coronavirus, e.g. without limitation severe acute respiratory syndrome coronavirus 2 (SARS-COV-2), comprising administering an effective amount of the pharmaceutical compositions described herein, or a method for treating or preventing a viral infection in a subject in need thereof, by providing plasma from a donor subject who has recovered from the viral infection, e.g. without limitation an infection with a coronavirus, e.g. without limitation SARS-COV-2, the plasma comprising IgG, IgM and/or IgA antibodies directed against the virus causing the infection and the donor subject having been treated with the recombinant human GM-CSF protein described herein to stimulate production of the antibodies; and administering the plasma to the subject in need thereof.

In aspects, there is provided a method of method of making a recombinant producing a composition comprising a recombinant human GM-CSF comprising: (a) obtaining a yeast cell transfected with a nucleic acid encoding a recombinant human GM-CSF, comprising an amino acid sequence having at least about 97% identity with SEQ ID NO: 2 and having a substitution or deletion at position N37, G38 and/or T39 or a position corresponding thereto, or an extract thereof; (b) purifying the GM-CSF from the transfected yeast cell using one or more HPLC columns, wherein the purification is in the absence of an organic solvent (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak; and (c) collecting the purified GM-CSF, the purified GM-CSF being substantially free of hyperglycosylated, e.g. hypermannosylated GM-CSF forms.

### BRIEF DESCRIPTION OF DRAWINGS

FIG. 1 shows Saccharomyces expression of GM-CSF as quantified by HPLC. The asterisks identifies the hypermannosylated GM-CSF peak.

FIG. 2 illustrates a western blot that was used to confirm protein identity.

FIG. 3 illustrates the reversed-phase HPLC assay that was used determine the percent distributions of rhu GM-CSF glycoforms.

FIG. 4 shows comparative chromatograms of BDS LEUKINE (Reference standard) grown in yeast versus CHO expression of rhu GM-CSF. BDS LEUKINE refers to wild type GM-CSF, SEQ ID NO: 1, without either of T39A or N37Q amino acid substitutions within the sequence.

FIG. 5 shows comparative size exclusion chromatograms of BDS LEUKINE (Reference standard) grown in yeast versus CHO expression of rhu GM-CSF.

FIG. 6 shows fermentation titer of rhu GM-CSF grown in yeast versus CHO cells.

FIG. 7 illustrates a comparative functional TF-1 bioassay that measure biological activity of rhu GM-CSF with mutations, T39A and N37Q, and LEUKINE (i.e. GM-CSF without T39A or N37Q).

FIG. 8 shows TF-1 assay data that measures biological activity of rhu GM-CSF with mutation N37Q grown in CHO cells as compared to 3 different batches of BDS LEUKINE that are stored using different conditions (Leukine BDS new material, Leukine BDS stored 12 month at 2-8° C., Leukine stored at −70° C. for 48 months).

### DETAILED DESCRIPTION

The present invention is based, in part, on the discovery that a single amino acid changes in the sequence of sargramostim, e.g. at positions, 37, 38, and/or 39 of sargramostim, or equivalents, yields a functional GM-CSF that does not require purification with organic solvents (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak that is observed with sargramostim.

### Compositions of GM-CSF

In an aspect, there is provided engineered GM-CSF proteins.

In embodiments, the engineered GM-CSF used in the practice of the invention includes any pharmaceutically safe and effective GM-CSF, or any derivative thereof having the biological activity of GM-CSF and the present substitutions and/or deletions.

In an embodiment, the engineered GM-CSF used in the practice of the subject methods is derived from recombinant human GM-CSF (rhu GM-CSF), such as sargramostim (LEUKINE). Sargramostim is a biosynthetic, yeast-derived, recombinant human GM-CSF, having of a single 127 amino acid glycoprotein that differs from endogenous human GM-CSF by having a leucine instead of a proline at position 23. Other natural and synthetic GM-CSFs, and derivatives thereof having the biological activity of natural human GM-CSF, may be equally useful in the practice of the invention.

In embodiments, the present recombinant human GM-CSF molecules are glycosylated. In embodiments, the present recombinant human GM-CSF molecules comprise one or more substitutions and/or deletions that impact the glycosylation of the GM-CSF.

Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993).

In an aspect, there is provided a recombinant human GM-CSF protein, comprising an amino acid sequence having at least about 90%, or at least about 91%, or at least about 92%, or at least about 93%, or at least about 94%, or at least about 95%, or at least about 96%, or at least about 97%, or at least about 98%, or at least about 99% identity, or 100% identity with SEQ ID NO: 1 or SEQ ID NO: 2 and having a substitution or deletion at position N37, E38 and/or T39 or a position corresponding thereto.

In embodiments, the amino acid at position N37 or a position corresponding thereto of the recombinant human GM-CSF is a polar and neutral of charge hydrophilic amino acid. In embodiments, the polar and neutral of charge hydrophilic amino acid is selected from glutamine (Q), serine (S), threonine (T), proline (P), and cysteine (C). in embodiments, the polar and neutral of charge hydrophilic amino acid is glutamine (Q).

In embodiments, the amino acid at position E38 or a position corresponding thereto of the recombinant human GM-CSF is a hydrophobic, aliphatic amino acid and/or is not a proline. In embodiments, the hydrophobic, aliphatic amino acid is selected from alanine (A), leucine (L), isoleucine (I), methionine (M), and valine (V).

In embodiments, the amino acid at position T39 or a position corresponding thereto of the recombinant human GM-CSF is a hydrophobic, aliphatic amino acid. In embodiments, the hydrophobic, aliphatic amino acid is selected from alanine (A), glycine (G), leucine (L), isoleucine (I), methionine (M), and valine (V). In embodiments, the hydrophobic, aliphatic amino acid is alanine (A). In embodiments, the amino acid at position T39 or a position corresponding thereto of the recombinant human GM-CSF is uncharged. In embodiments, the amino acid at position T39 or a position corresponding thereto of the recombinant human GM-CSF is not Glu glutamic acid (E).

In embodiments, there is provided a recombinant human GM-CSF protein, comprising an amino acid sequence having at least about 90%, or at least about 91%, or at least about 92%, or at least about 93%, or at least about 94%, or at least about 95%, or at least about 96%, or at least about 97%, or at least about 98%, or at least about 99% identity, or 100% identity with SEQ ID NO: 1 or SEQ ID NO: 2 and having one or more of N37 deletion, N37Q, N37S, N37T, N37P, N37C, E38 deletion, E38A, E38L, E381, E38M, E38V. T39 deletion, T39A, T39G, T39L, T391, T39M, and T39V, or corresponding mutation.

In embodiments, the recombinant human GM-CSF has an amino acid sequence of SEQ ID NO: 1 or SEQ ID NO: 2, or a variant of about 90%, or about 93%, or about 95%, or about 97%, or about 98% identity thereto with the present substitutions and/or deletions.

In embodiments, the GM-CSF is one of molgramostim, sargramostim, and regramostim having the present substitutions and/or deletions.

Without wishing to be bound by theory, the core of hGM-CSF consists of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present invention and these helices are to be retained in a functional fragments or deletion variants of hGM-CSF for use in this invention.

Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

Accordingly, in embodiments, this information may inform a skilled artisan with regard to acceptable variations in the amino acid sequences.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

### Glycoforms

In embodiments, the present recombinant human GM-CSF molecules comprise a plurality of molecular forms. In embodiments, the molecular forms are selected from non-glycosylated, O-glycosylated, N-glycosylated and N+O glycosylated forms.

In embodiments, the recombinant human GM-CSF is substantially free of hyperglycosylated, e.g. hypermannosylated forms. In embodiments, the recombinant human GM-CSF has about, or less than about, 10% hypermannosylated forms after purification, or about, or less than about, 5% hypermannosylated forms after purification, or about, or less than about, 3% hypermannosylated forms after purification, or about, or less than about, 2% hypermannosylated forms after purification, or about, or less than about, 1% hypermannosylated forms after purification.

In embodiments, the recombinant human GM-CSF has less hypermannosylated forms than wild type human GM-CSF and/or sargramostim, when expressed and purified in the same manner. In embodiments, the recombinant human GM-CSF has about 100%, or about 90%, or about 80%, or about 70%, or about 60%, or about 50%, or about 40%, or about 30%, or about 20%, or about 10% less hypermannosylated forms than wild type human GM-CSF and/or sargramostim, when expressed and purified in the same manner. In embodiments, the amount of hypermannosylated forms can be detected as known in the art, e.g., without limitation, chromatographic methods (e.g. size resolution (e.g. via molecular weight and/or retention times in a column and/or fluorescent labeling (e.g. using 2-aminobenzoic acid (2-AA), 2-aminobenzamide (2-AB), and 2-aminopyridine (2-AP), anion exchange chromatography, and the like), mass spectrometry, SDS-PAGE/staining (e.g. gel-staining procedures based on the periodic acid-Schiff (PAS) reaction), affinity-based methods, such as the use of saccharide-binding proteins (e.g. lectins), enzyme-based methods, antibody-based methods, release assays (e.g. enzymatic cleavage or chemical removal of glycans or chemical derivatization), capillary electrophoresis, and eastern blot.

In embodiments, the present recombinant human GM-CSF is suitable for purification that is less complicated than what is used for sargramostim.

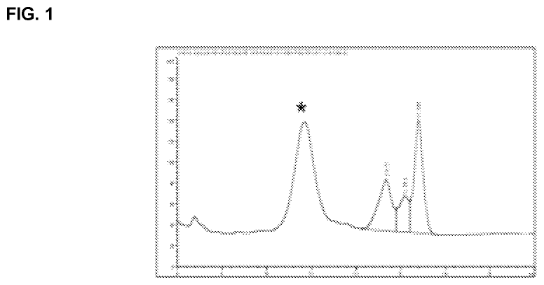

In embodiments, the present mutant GM-CSF proteins resolves, e.g., via HPLC, as three glycoforms and lack a hyperglycosylated form. Contrast with FIG. 1, which shows four peaks in GM-CSF, as resolved on HPLC—the asterisks identifies the hypermannosylated GM-CSF peak.

In embodiments, the present GM-CSF proteins are purifiable without a hyperglycosylated species, without the need for one or more organic solvents (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak. In embodiments, the present GM-CSF proteins are purifiable without a hyperglycosylated species, without the use of acetonitrile, trifluoroacetic acid (TFA), pyridine, acetic acid and/or N-propanol. In embodiments, the present GM-CSF proteins are purifiable without a reversed phase C4 HPLC column used for the purposes of purification and/or removal of the hyperglycosylated peak.

In embodiments, the present recombinant human GM-CSF resolves as three peaks when quantified by reversed phase high-performance liquid chromatography (RP-HPLC). In embodiments, the recombinant human GM-CSF provides no substantial peaks at a retention time of less than about 20 minutes when quantified by reversed phase high-performance liquid chromatography (RP-HPLC).

In embodiments, the present recombinant human GM-CSF comprises more than one species (e.g. glycoforms). In embodiments, none of the species have a molecular weight of greater than about 20 kDa.

In embodiments, the recombinant human GM-CSF is substantially free of hypermannosylated forms when purified without the use of organic solvents (e.g. without limitation, acetonitrile, trifluoroacetic acid (TFA), pyridine, acetic acid and/or N-propanol) (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak.

In embodiments, the recombinant human GM-CSF produced in a mammalian cell, such as a Chinese Hamster Ovary (CHO) cell is monomeric with heterogeneous glycosforms with both glycosylated and aglycosylated isoforms.

### Functional Properties of the Recombinant GM-CSF

In embodiments, the present recombinant human GM-CSF molecules, with the present substitutions and/or deletions, functionally similar to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold of the assayed functional parameter). In embodiments, the functional parameters of GM-CSF can be detected by assays known in the art, e.g., without limitation, proliferation assays using cells such as TF-1 cell lines, primary bone marrow cells, biochemical assays such as ILITE (EAGLE) GM-CSF (luciferase under the control of GM-CSF promoter), cell survival assays e.g. myeloid cell survival assay, cell differentiation assays and co-culture experiments

In embodiments, the present recombinant human GM-CSF molecules, with the present substitutions and/or deletions, bind and/or activate the granulocyte-macrophage colony stimulating factor receptor (GM-CSF-R-alpha or CSF2R). In embodiments, the present recombinant human GM-CSF molecules, with the present substitutions and/or deletions, bind and/or activate the granulocyte-macrophage colony stimulating factor receptor (GM-CSF-R-alpha or CSF2R) at an affinity, efficacy, and/or bioactivity that is comparable to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold). Assays for GM-CSF binding and activation are known in the art. Non-limiting examples of such assays include, for example, radioligand assays or non-radioligand assays (e.g. immunoprecipitation (IP), enzyme-linked immunosorbent assay (ELISA), western blot, fluorescence polarization (FP). fluorescence resonance energy transfer (FRET), surface plasmon resonance (SPR), and radioimmunoassay (RIA). The binding kinetics also can be assessed by standard assays known in the art, such as by Biacore analysis. Whole cell ligand-binding assays, and cell-free assay systems using soluble GM-CSF receptor alpha (sGMRa) may also be used. Some other types of assays that may be used include, receptor-binding, or saturation binding, or competitive binding assays using radio-iodinated GM-CSF, as well as cell proliferation assays.

In embodiments, the present recombinant human GM-CSF molecules can be assayed using one or more cell-based activity bioassays, e.g. using a GM-CSF dependent human cell-line proliferation assay, e.g. using TF-1, M-07e, HU-3, M-MOK, MB-02, GM/SO, F-36P, GF-D8, ELF-153, AML-193, MUTZ-3, OCI-AML5, OCI-AML6, OCI-AML1, SKNO-1, UCSD-AML1 and UT-7.

In embodiments, the potency of the present recombinant human GM-CSF molecules is measured using a bioassay employing TF-1 cells, a human erythroid leukemia cell line that proliferates in response to GM-CSF. The details of this assay are known in the art. For instance, a reference standard, control and test samples are serially diluted in triplicate in assay media and added to three separate 96-well plates. TF-1 cells in suspension are then added and the mixture is incubated at 37ºC for 69.5-72 hours. Following the addition of a fluorescent dye (e.g. ALAMARBLUE), the plates are incubated at 37° C. for 6.6-8 hours. TF-1 cell proliferation is then measured in a fluorescent microplate reader.

In embodiments, the present recombinant human GM-CSF molecules have roughly the specific same activity as a recombinant human GM-CSF lacking the mutations (e.g. as assayed using a bioassay employing TF-1 cells).

In embodiments, the recombinant human GM-CSF produced in a mammalian cell, such as a Chinese Hamster Ovary (CHO) cell is functionally enhanced as compared to a recombinant human GM-CSF produced in a yeast cell.

In embodiments, the GM-CSF-R-alpha at which binding and/or activation occurs is expressed on the surface of a cell. In embodiments, the cell is a hematopoietic progenitor cell. In embodiments, the hematopoietic progenitor cell is an immune cell. In embodiments, the hematopoietic progenitor cell is irradiated.

In embodiments, the immunogenicity of the present recombinant human GM-CSF molecules, with the present substitutions and/or deletions is comparable to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold). In embodiments, immunogenicity is assayed using methods known in the art. Non-limiting examples include detection of one or more anti-GM-CSF binding antibodies as assessed by, e.g. screening assays such as direct or indirect or bridging ELISA, electrochemiluminescence, bead-based chemiluminescence assays, radioimmunoprecipitation assay, surface plasma resonance and bio layer interferometry, as well as cell based luciferase reporter gene neutralizing antibody assay.

In embodiments, the cell recombinant human GM-CSF is soluble.

### Nucleic Acids and Host Cells

In embodiments, there is provided a nucleic acid molecule encoding the recombinant human GM-CSF described herein. In embodiments, the nucleic acid molecule has a codon-optimized sequence.

In embodiments, there is provided a non-human host cell expressing the nucleic acid molecule described herein.

In embodiments, the host cell is a yeast, mammalian, bacterial, insect, algae, or plant cell.

In embodiments, the yeast cell is a non-methylotrophic yeast cell. In embodiments, the host cell is a Saccharomyces cerevisiae cell.

In embodiments, there is provided a CHO cell expressing the nucleic acid molecule described herein.

### Pharmaceutical Compositions and Formulations

In embodiments, there is provided a pharmaceutical composition comprising a recombinant human GM-CSF described herein and a pharmaceutically acceptable excipient or carrier.

Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, dessicated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid

Where necessary, the present pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional therapeutic agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional therapeutic agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In specific embodiments, the GM-CSF (and/or additional therapeutic agents) is administered via an intravenous route.

In one embodiment, the pharmaceutical compositions (and/or additional therapeutic agents) described herein are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any pharmaceutical compositions (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Any inventive pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N, N-di-lower alkyl-N-(hydroxyl-lower alkyl)-amines, such as N, N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Methods of Use

In an aspect, there is provided a method of treating a patient or subject who is undertaking or has undertaken a cancer therapy, or who is undertaking or has undertaken a bone marrow transplant, and/or who had been acutely exposed to myelosuppressive doses of radiation; the method comprising administering to the patient a therapeutically effective amount of the present recombinant human GM-CSF protein or a pharmaceutical composition thereof. In embodiments, the patient is treated by modulating clonal expansion, survival, differentiation and activation state of hematopoietic progenitor cells. In embodiments, the patient is treated by modulating a myelomonocytic cell lineage, by promoting the proliferation of megakaryocytic and erythroid progenitors. In embodiments, the patient is treated by modulating hematopoietic progenitor cells, by stimulating the survival, proliferation and activation of neutrophils, macrophages and/or dendritic cells. In embodiments, the patient is treated following bone marrow transplant by modulating hematopoietic progenitor cells, by stimulating the survival, proliferation and activation of neutrophils, macrophages and/or dendritic cells.

In an aspect, there is provided a therapeutic method comprising administering to a patient a therapeutically effective amount of the present recombinant human GM-CSF protein or a pharmaceutical composition thereof or contacting cells with an effective amount of the pharmaceutical composition described herein and administering therapeutically effective amount of the cells, wherein the therapy: accelerates neutrophil recovery and/or to reduce the incidence of infections following induction chemotherapy; mobilizes hematopoietic progenitor cells into peripheral blood for collection by leukapheresis and transplantation; accelerates of myeloid reconstitution following autologous or allogeneic bone marrow or peripheral blood progenitor cell transplantation; treats delayed neutrophil recovery or graft failure after autologous or allogeneic bone marrow transplantation; and/or treats hematopoietic syndrome of acute radiation syndrome (H-ARS).

In an aspect, there is provided a method for treating an infection with a virus, comprising: administering an effective amount of a composition comprising the present recombinant human GM-CSF protein or a pharmaceutical composition comprising the same to a patient in need thereof.

In embodiments, the viral infection is an influenza infection, optionally selected from Type A, Type B, Type C, and Type D influenza virus infection.

In embodiments, the viral infection is a coronavirus infection. In embodiments, the coronavirus is a betacoronavirus, optionally selected from severe acute respiratory syndrome coronavirus 2 (SARS-COV-2), SARS-COV, Middle East respiratory syndrome-corona virus (MERS-COV), HCoV-HKU1, and HCoV-OC43. In embodiments, the coronavirus is an alphacoronavirus, optionally selected from HCoV-NL63 and HCoV-229E.

The coronavirus is a member of the family Coronaviridae, including betacoronavirus and alphacoronavirus respiratory pathogens that have relatively recently become known to invade humans. The Coronaviridae family includes such betacoronavirus as Severe acute respiratory syndrome coronavirus 2 (SARS-COV-2), SARS-COV, Middle East Respiratory Syndrome-Corona Virus (MERS-COV), HCoV-HKU1, and HCoV-OC43. Alphacoronavirus includes, e.g., HCoV-NL63 and HCoV-229E.

Coronaviruses invade cells through “spike” surface glycoprotein that is responsible for viral recognition of Angiotensin Converting Enzyme 2 (ACE2), a transmembrane receptor on mammalian hosts that facilitate viral entrance into host cells. Zhou et al., A pneumonia outbreak associated with a new coronavirus of probable bat origin. Nature 2020. A new coronavirus infection 2019 (COVID-19), caused by

SARS-COV-2 is a new virus thought to be originated from the bat. COVID-19 causes severe respiratory distress and this RNA virus strain has been the cause of the recent outbreak that has been declared a major threat to public health and worldwide emergency. Phylogenetic analysis of the complete genome of SARS-COV-2 revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020

The SARS-COV-2 is an enveloped, single stranded, RNA virus that encodes a “spike” protein, also known as the S protein, which is a surface glycoprotein that mediates binding to a cell surface receptor; an integral membrane protein; an envelope protein, and a nucleocapsid protein. The S protein, comprising S1 subunit and S2 subunit, is a trimeric class I fusion protein that exists in a prefusion conformation that undergoes a structural rearrangement to fuse the viral membrane with the host-cell membrane. See, e.g., Li, F. Structure, Function, and Evolution of Coronavirus Spike Proteins. Annu. Rev. Virol. 3: 237-261(2016), which is incorporated herein by reference in its entirety. The structure of the SARS-COV-2 spike protein in the prefusion conformation has been discovered. See Daniel et al., Cryo-EM structure of the SARS-COV-2 spike in the prefusion conformation. Science, 19 Feb. 2020, which is incorporated herein by reference in its entirety.

Phylogenetic analysis of the complete genome of SARS-COV-2 (GenBank Accession No.: MN908947) revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020, which is incorporated herein by reference in its entirety.

The SARS-COV-2 has a spike surface glycoprotein, membrane glycoprotein M, envelope protein E, and nucleocapsid phosphoprotein N. The complete genome of the SARS-COV-2 coronavirus (29903 nucleotides, single-stranded RNA) is described in the NCBI database as GenBank Reference Sequence: MN908947. The coronavirus protein can be selected from the group consisting of: coronavirus spike protein (GenBank Reference Sequence: QHD43416), coronavirus membrane glycoprotein M (GenBank Reference Sequence: QHD43419), coronavirus envelope protein E (GenBank Reference Sequence: QHD43418), and coronavirus nucleocapsid phosphoprotein E (GenBank Reference Sequence: QHD43423).

In embodiments, the method prevents or mitigates development of acute respiratory distress syndrome (ARDS) in the patient.

In embodiments, the coronavirus is SARS-COV-2. In embodiments, the patient is afflicted with COVID-19. In embodiments, the patient is afflicted with one or more of fever, cough, shortness of breath, diarrhea, upper respiratory symptoms, lower respiratory symptoms, pneumonia, and acute respiratory syndrome.

In embodiments, the patient is hypoxic. In embodiments, the patient is afflicted with respiratory distress. In embodiments, the method improves oxygenation in the patient. In embodiments, the method prevents or mitigates a transition from respiratory distress to cytokine imbalance in the patient. In embodiments, the method reverses or prevents a cytokine storm. In embodiments, the method reverses or prevents a cytokine storm in the lungs or systemically. In embodiments, the cytokine storm is selected from one or more of systemic inflammatory response syndrome, cytokine release syndrome, macrophage activation syndrome, and hemophagocytic lymphohistiocytosis. In embodiments, the method reverses or prevents excessive production of one or more inflammatory cytokines. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7.

In embodiments, the method causes a decrease in viral load in the patient relative to before treatment.

In an aspect, there is provided a method for treating or preventing a viral infection in a subject in need thereof, comprising providing plasma from a donor subject who has recovered from the viral infection, the plasma comprising IgG, IgM and/or IgA antibodies directed against the virus causing the infection and the donor subject having been treated with the recombinant human GM-CSF protein described herein to stimulate production of the antibodies; and administering the plasma to the subject in need thereof. In an aspect, there is provided a method for treating or preventing a viral infection in a subject in need thereof, comprising: administering the recombinant human GM-CSF protein described herein to a donor subject who has recovered from the viral infection; isolating plasma from the donor subject, the plasma comprising IgG, IgM and/or IgA antibodies directed against the virus causing the infection; and administering the plasma to the subject in need thereof.

In embodiments, such methods provide passive immunization against the virus to the subject in need thereof.

In embodiments, the IgG, IgM and/or IgA antibodies specifically bind to a viral antigen. In embodiments, the IgG, IgM and/or IgA antibodies neutralize the virus. In embodiments, the IgG, IgM and/or IgA antibodies prevent or diminish infection of a cell by the virus.

In embodiments, the viral infection is selected from a betacoronavirus infection, optionally selected from severe acute respiratory syndrome coronavirus 2 (SARS-COV-2), severe acute respiratory syndrome coronavirus (SARS-COV-1), Middle East Respiratory Syndrome-Corona Virus (MERS-COV), HCoV-HKU1, and HCoV-OC43 infection. In embodiments, the viral infection is selected from an alphacoronavirus infection, optionally selected from HCoV-NL63 and HCoV-229E infection.

In embodiments, the betacoronavirus infection is severe acute respiratory syndrome (SARS).

In embodiments, the betacoronavirus infection is, or is associated with, coronavirus disease 2019 (COVID-19).

In embodiments, the viral infection is an influenza infection, optionally selected from Type A, Type B, Type C, and Type D influenza virus infection. In embodiments, the influenza infection is pandemic 2009 influenza A (H1N1) or avian influenza A (H5N1).

In embodiments, donor subject has tested positive for the viral infection prior to recovery. In embodiments, the donor subject has resolution of viral infection symptoms prior to donation. In embodiments, the donor subject has tested positive for antibodies directed against the virus using a serological test. In embodiments, the donor subject demonstrates measurable neutralizing antibody titers. In embodiments, the neutralizing antibody titers are at least about 1:160. In embodiments, the plasma is isolated from a blood sample from the donor subject. In embodiments, the plasma is isolated via plasmapheresis. In embodiments, the plasma comprises a therapeutically effective amount of the IgG, IgM and/or IgA antibodies directed against the virus causing the infection.

### Combination Therapy and Additional Therapeutic Agents

In various embodiments, the pharmaceutical composition of the present invention is co-administered in conjunction with additional agent(s). Co-administration can be simultaneous or sequential.

In one embodiment, the additional therapeutic agent and the GM-CSF of the present invention are administered to a subject simultaneously. The term “simultaneously” as used herein, means that the additional therapeutic agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the GM-CSF can be by simultaneous administration of a single formulation (e.g., a formulation comprising the additional therapeutic agent and the GM-CSF composition) or of separate formulations (e.g., a first formulation including the additional therapeutic agent and a second formulation including the GM-CSF composition).

Co-administration does not require the therapeutic agents to be administered simultaneously, if the timing of their administration is such that the pharmacological activities of the additional therapeutic agent and the GM-CSF overlap in time, thereby exerting a combined therapeutic effect. For example, the additional therapeutic agent and the targeting moiety, the GM-CSF composition can be administered sequentially. The term “sequentially” as used herein means that the additional therapeutic agent and the GM-CSF are administered with a time separation of more than about 60 minutes. For example, the time between the sequential administration of the additional therapeutic agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the additional therapeutic agent or the GM-CSF composition may be administered first.

Co-administration also does not require the therapeutic agents to be administered to the subject by the same route of administration. Rather, each therapeutic agent can be administered by any appropriate route, for example, parenterally or non-parenterally.

In some embodiments, the GM-CSF described herein acts synergistically when co-administered with another therapeutic agent. In such embodiments, the targeting moiety, the GM-CSF composition and the additional therapeutic agent may be administered at doses that are lower than the doses employed when the agents are used in the context of monotherapy.

In some embodiments, the additional therapeutic agent is an anti-viral drug.

In some embodiments, the additional therapeutic agent is selected from drugs including antivirals such as remdesivir, favipiravir, oseltamivir, baloxavir, galidesivir, amprenavir, tipranavir, saquinavir, nelfinavir, indinavir, darunavir, atazanavir, emetine, lopinavir and/or ritonavir, arbidol and lopinavir/ritonavir, and/or ribavirin, darunavir and cobicistat, and/or IFN-beta-1b, β-D-N4-hydroxycytidine (NHC) such as EIDD-1931 or EIDD-2801 or EIDD-2801; immunomodulators such as glucocorticoids, IFN-α 2a, IFN-α 2b, IFN-b, pegylated IFN-g, baricitinib, sirolimus, clazakizumab, canakinumab, XPro1595, tocilizumab, sarilumab, siltuximab, adalimumab, eculizumab, ivermectin, anakinra, prezcobix, xiyanping, fingolimod, methylprednisolone, leronlimab, thalidomide, MK-2206, nicolasamide, nitazoxamide, chloroquine or hydroxychloroquine; antibiotics such as carrimycin, brilacidin, azithromycin, valinomycin, angiotension inhibitors/antagonists like rhACE2/GSK2586881/APN01, losartan, eprosartan, telmisartan, valsartan; serine protease inhibitor including camostat mesylate, nafamostat other drugs such as bromhexine, aprotinin, chlorpromazine, zotatifin, methotrexate, lenalidomide, anti-VEGF-A and Intravenous Immunoglobulin (IVIG). For instance, in embodiments, any of these additional therapeutic agents find use in the context of a SARS-COV-2 infection.

In some embodiments, the additional therapeutic agent is selected from favipiravir, laninamivir octanoate, peramivir, zanamivir, oseltamivir phosphate, baloxavir marboxil, umifenovir, urumin amantadine hydrochloride, rimantadine hydrochloride, adapromine, LASAG/BAY81-87981, celecoxib, etanercept, metformin, gemcitabine, dapivirine, trametinib, lisinopril, naproxen, nalidixic acid, dorzolamide, ruxolitinib, midodrine, diltiazem; statins including atorvastatin, nitazoxanide; PPAR antagonists including gemfibrozil. For instance, in embodiments, any of these additional therapeutic agents find use in the context of a influenza infection.

### Methods of Making

In aspects, there is provided a method of method of making a recombinant producing a composition comprising a recombinant human GM-CSF comprising: (a) obtaining a cell transfected with a nucleic acid encoding a recombinant human GM-CSF, comprising an amino acid sequence having at least about 90%, at least about 93%, at least about 95%, at least about 97%, at least about 98%, or at least about 99% identity with SEQ ID NO: 1 or SEQ ID NO: 2 and having a substitution or deletion at position N37, G38 and/or T39 or a position corresponding thereto as described herein, or an extract thereof; (b) purifying the GM-CSF from the transfected yeast cell using one or more HPLC columns, wherein the purification is in the absence of an organic solvent (e.g. without limitation, acetonitrile, trifluoroacetic acid (TFA), pyridine, acetic acid and/or N-propanol); and (c) collecting the purified GM-CSF, the purified GM-CSF being substantially free of hyperglycosylated, e.g. hypermannosylated GM-CSF forms.

In embodiments, the cell is a prokaryotic or eukaryotic host cell, e.g. yeast, mammalian, bacterial, insect, algae, or plant cell.

Suitable prokaryotic host cells include bacterial cells (e.g., E coli, Bacillus subtilis, Mycobacterium spp., M. tuberculosis or other appropriate bacterial cells), and archaeal cells (e.g. Methanococcus jannaschii and Methanococcus maripaludis).

In some embodiments, the host cell of the present disclosure is a eukaryotic host cell. Suitable eukaryotic host cells include, but are not limited to: fungal cells, algal cells, insect cells, animal cells (e.g., mammalian cells, avian cells, and fish cells), and plant cells.

Suitable fungal host cells include, but are not limited to: Ascomycota, Basidiomycota, Deuteromycota, Zygomycota, Fungi imperfecti.

Suitable yeast host cells include, but are not limited to: Candida, Hansenula, Saccharomyces, Schizosaccharomyces, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Kluyveromyces lactis, Candida albicans, or Yarrowia lipolytica.

Suitable filamentous fungi host cells include, for example, any filamentous forms of the subdivision Eumycotina and Oomycota. In embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from the group consisting of A. nidulans, A. oryzae, A. sojae, and Aspergilli of the A. niger Group. In an embodiment, the filamentous fungus is Aspergillus niger.

In embodiments, the cell is a yeast cell, e.g. without limitation Saccharomyces cerevisiae.

In embodiments, the cell is a mammalian cell, e.g. without limitation Chinese Hampster Ovary (CHO) cell.

In embodiments, production of the recombinant protein in a mammalian cell increases expression levels of the recombinant protein, as compared to production using a non-mammalian cell.

In embodiments, the method further comprises formulating the purified GM-CSF for injection, e.g. subcutaneous or intravenous injection.

### Sequences

SEQ ID NO: 1 is wild type GM-CSF. The sites of the present substitutions of deletions are indicated by underlining and bold:

APARSPSPSTQPWEHVNAIQEARRLLNLSRDTAAEMNETVEVISEMFDL
QEPTCLQTRLELYKQGLRGSLTKLKGPLTMMASHYKQHCPPTPETSCAT
QIITFESFKENLKDFLLVIPFDCWEPVQE

SEQ ID NO: 2 is sargramostim. The sites of the present substitutions of deletions are indicated by underlining and bold:

APARSPSPSTQPWEHVNAIQEALRLLNLSRDTAAEMNETVEVISEMFDL
QEPTCLQTRLELYKQGLRGSLTKLKGPLTMMASHYKQHCPPTPETSCAT
QIITFESFKENLKDFLLVIPFDCWEPVQE

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with an agent effective for the treatment of a coronavirus infection is an amount that is effective for treating or mitigating a coronavirus infection.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

This invention is further illustrated by the following non-limiting examples.

### EXAMPLES

### Example 1: Cloning and Purification of GM-CSF Mutants

Fermentation and processing of the protein GM-CSF product, without or without the present mutations, was undertaken in the absence of organic solvents that is used to remove the hyperglycosylation peak (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak.

Importantly, organic solvents and a reversed phase C4 HPLC are used in the production of GM-CSF that lacks the present mutations, e.g. to remove a large hyperglycosylated form peak from the three glycoforms (see FIG. 1). Such organic solvents were not used for the mutants described herein, which obviated the need to use a reversed phase C4 HPLC intended to remove these organic solvents.

### Example 2: Biochemical Assays of GM-CSF Mutants

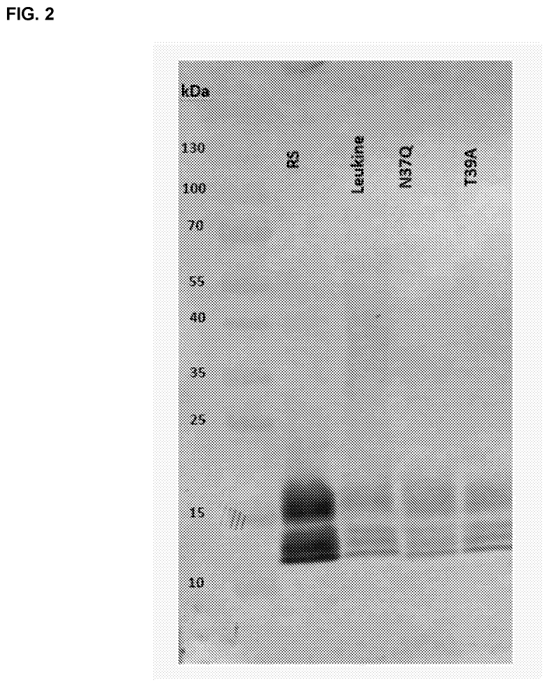

Biochemical identity of rhu GM-CSF was confirmed by Coomassie stained sodium dodecylsulfate polyacrylamide gel electrophoresis and Western Blot analysis (FIG. 2). Purified rhu GM-CSF protein samples were diluted in Tris-Glycine SDS Sample Buffer. Diluted rhu GM-CSF samples, as well as a Reference Standard, were denatured by heating at 100° C. for 5 minutes in a heat block. The Reference Standard and rhu GM-CSF samples were loaded onto a pre-cast 20% SDS-PAGE gel alongside a molecular weight marker. The loaded gel was electrophoresed at a constant voltage to separate proteins, and subsequently incubated in Coomassie G-250 stain to visualize proteins. rhu GM-CSF migrated as 3 bands on SDS-PAGE. The supernatants were run on SDS-PAGE (4 ug/lane) and compared to a molecular weight ladder and a reference standard.

Western Blot analysis was performed to confirm protein identity (FIG. 2). Purified rhu GM-CSF protein samples and a Reference Standard were prepared for SDS-PAGE as previously described. The Reference Standard and rhu GM-CSF samples were loaded onto a pre-cast 20% SDS-PAGE alongside a pre-stained molecular weight marker. The loaded gel was electrophoresed at a constant voltage to separate proteins, which were subsequently transferred from the gel to a nitrocellulose membrane. The nitrocellulose membrane was then blotted with mouse anti-human GM-CSF monoclonal antibodies, followed by goat anti-mouse IgG conjugated to alkaline phosphatase for immunodetection. An alkaline phosphatase color developer was used to visualize the bands. All supernatants blotted positive for GM-CSF (positive bands in the GM-CSF region as compared to the Ref Standard) (FIG. 2).

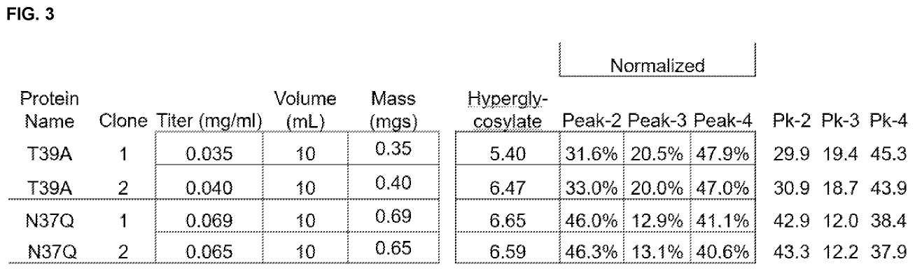

Reversed-phase HPLC was used to determine the percent distributions of rhu GM-CSF glycoforms (FIG. 3). Neither organic solvents nor a reversed phase C4 HPLC column was used. The single point mutants T39A (Alanine substitution for Threonine at position 39) and N37Q (Glutamine substitution for Asparagine at position 37) were tested in a reversed phase HPLC assay to determine titer and glycoforms. This assay generates titer (mg/ml) using a calibration curve made of reference standard. This procedure resolved rhu GM-CSF glycosylated variants into three main glycoform groups. Test sample rhu GM-CSF concentration results were interpolated from a six-level external standard calibration curve prepared from a GM-CSF reference standard. Four peaks of interest were integrated and quantitated; the composition of each is described below:
Peak 1=GM-CSF related impurity (oxidation).
Peak 2=N- and (N+O) linked glycoforms
Peak 3=O-linked glycoforms
Peak 4=Non-glycosylated GM-CSF
   The assay separates residual process components and the hyperglycosylated material away from the product peaks to get a glycoform profile (peak 2-4). The product peaks were reported as normalized as to not factor in the hyperglycosylated material. T39A had ˜6% hyperglycosylation and N37Q had ˜6.6%. The two samples have a lower hyperglycosylated percentage GM-CSF without the mutation (˜40%, See FIG. 1). Hyperglycosylated material is typically removed via chromatography with organic solvents (e.g. without limitation, acetonitrile, trifluoroacetic acid (TFA), pyridine, acetic acid and/or N-propanol) and a reversed phase C4 HPLC column used for the purposes of purification, and these mutants are of interest as, inter alia, they may be able to eliminate a process step which would save time, resources and use less hazardous solvents in the purification process.

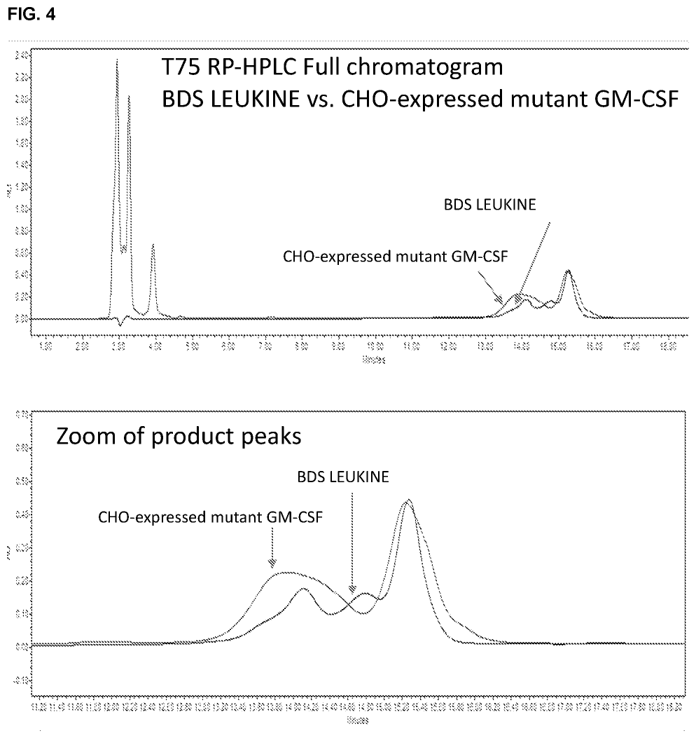

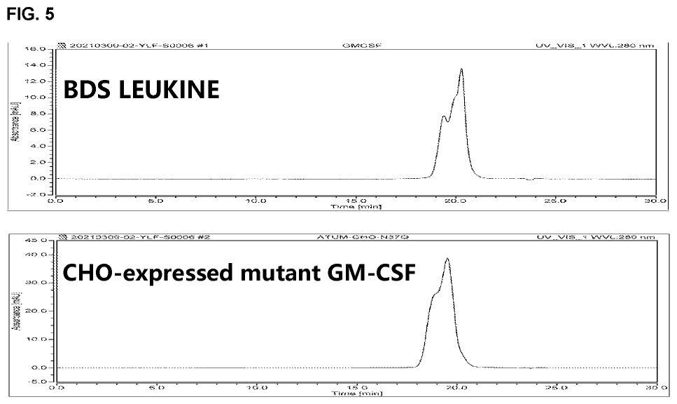

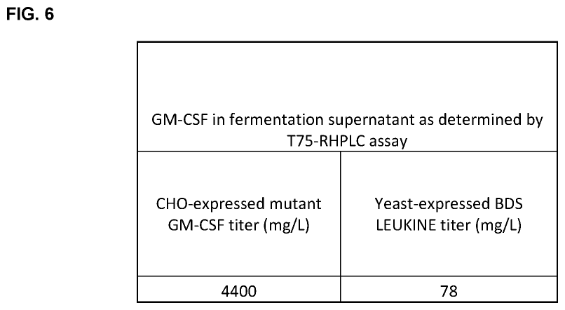

Reversed-phase HPLC was also used to determine the GM-CSF glycoforms for BDS LEUKINE as compared to CHO-expressed mutant GM-CSF (FIG. 4). Size exclusion chromatography analysis was also used to determine molar mass distribution (FIG. 5). The single point mutants N37Q (Glutamine substitution for Asparagine at position 37) and BDS LEUKINE were tested in a reversed phase HPLC assay and SEC analysis to determine titer (FIG. 6) and glycoforms (FIG. 4 and FIG. 5). BDS LEUKINE is produced using Saccromyces, a yeast cell, and the mutant GM-CSF was produced using a mammalian CHO cell. This assay generates titer (mg/ml) using a calibration curve made of reference standard. This procedure resolved rhu GM-CSF glycosylated variants into three main glycoform groups. Test sample rhu GM-CSF concentration results were interpolated from a six-level external standard calibration curve prepared from a GM-CSF reference standard. Similar to BDS LEUKINE, CHO-expressed mutant GM-CSF were monomeric (FIG. 5) and both samples showed multiple glycoforms (FIG. 4). Production in CHO cells increased the titer of the recombinant protein as compared to production in yeast (FIG. 6). BDS LEUKINE refers to wild type GM-CSF, SEQ ID NO: 1, without either of T39A or N37Q amino acid substitutions within the sequence.

### Example 3: Functional Assays of GM-CSF Mutants

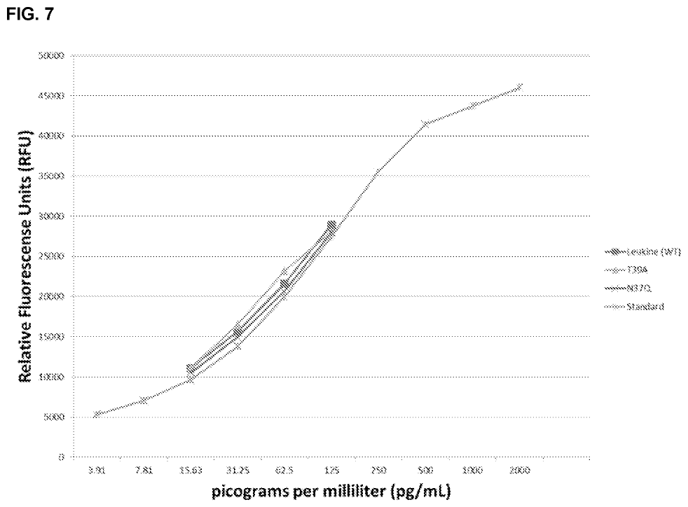

Potency of rhu GM-CSF was measured using a bioassay employing TF-1 cells, a human erythroid leukemia cell line that proliferates in response to GM-CSF (FIG. 7). A Reference Standard (rhu GM-CSF), control and test samples were serially diluted in triplicate in assay media and added to three separate 96-well plates. TF-1 cells in suspension were then added and the mixture is incubated at 37ºC for 69.5-72 hours. Following the addition of a fluorescent dye (ALAMARBLUE, THERMO), the plates were incubated at 37ºC for 6.6-8 hours. TF-1 cell proliferation was then measured in a fluorescent microplate reader. The single point T39A and N37Q, and GM-CSF without T39A or N37Q mutations (LEUKINE) were tested in the TF-1 bioassay. Specific activity (SA) in international units per milligram (IU/mg) were determined from a standard curve using a reference standard (sargramostim—rhuGM-CSF). Both mutants showed activity that was similar to LEUKINE, and SA's were determined to be 9.13×10⁶ (T39A) and 7.48×10⁶ (N37Q) as compared to 8.14×10⁶ IU/mg for LEUKINE:

| Sample ID | Specific Activity (IU/mg) |
|---|---|
| Leukine | 8.14 × 10⁶ |
| T39A | 9.13 × 10⁶ |
| N37Q | 7.48 × 10⁶ |

Surprisingly, these amino acid substitutions, despite being dramatic (e.g., without wishing to be bound by theory, for T39A—significantly reducing side chain length and for N37Q—increasing side chain length) do not affect biological activity, while allowing for easier manufacturability.

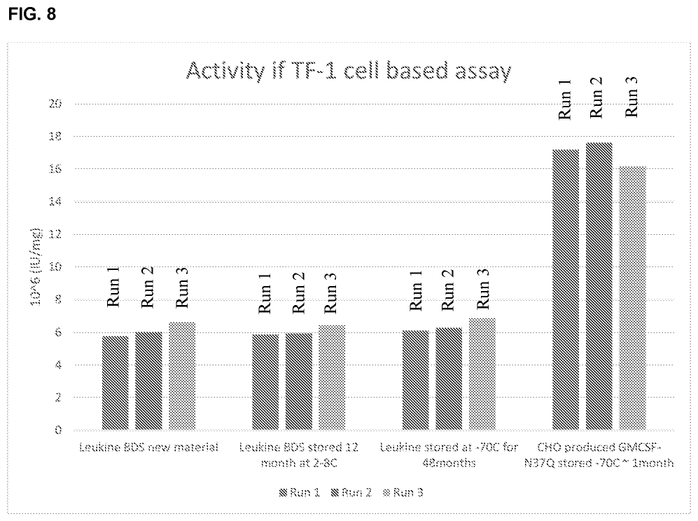

Further, potency of mutant rhu GM-CSF was measured using a bioassay employing TF-1 cells, a human erythroid leukemia cell line that proliferates in response to GM-CSF. Mutant rhu GM-CSF with a single amino acid at position N37 grown in CHO cells significantly enhanced activity (FIG. 8) of the recombinant protein as compared to 3 different batches of BDS LEUKINE grown in yeast cell, Saccromyces, stored using different conditions (Leukine BDS new material, Leukine BDS stored 12 months at 2-8° C., Leukine stored at −70° C. for 48 months). BDS LEUKINE refers to wild type GM-CSF, SEQ ID NO: 1, without either of T39A or N37Q amino acid substitutions within the sequence.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A composition comprising a recombinant human granulocyte macrophage-colony stimulating factor (GM-CSF) protein, comprising an amino acid sequence having at least about 97% identity with SEQ ID NO: 1 or SEQ ID NO: 2 and having a substitution or deletion at position N37, E38 and/or T39 or a position corresponding thereto.

2. The recombinant protein of claim 1, wherein the amino acid at position N37 or a position corresponding thereto is a polar and neutral of charge hydrophilic amino acid.

3. The recombinant protein of claim 2, wherein the polar and neutral of charge hydrophilic amino acid is selected from glutamine (Q), serine (S), threonine (T), proline (P), and cysteine (C).

4. The recombinant protein of claim 3, wherein the polar and neutral of charge hydrophilic amino acid is glutamine (Q).

5. The recombinant protein of claim 1, wherein the amino acid at position E38 or a position corresponding thereto is a hydrophobic, aliphatic amino acid and/or is not a proline.

6. The recombinant protein of claim 5, wherein the hydrophobic, aliphatic amino acid is selected from alanine (A), leucine (L), isoleucine (I), methionine (M), and valine (V).

7. The recombinant protein of claim 1, wherein the amino acid at T39 or a position corresponding thereto is a hydrophobic, aliphatic amino acid.

8. The recombinant protein of claim 7, wherein the hydrophobic, aliphatic amino acid is selected from alanine (A), glycine (G), leucine (L), isoleucine (I), methionine (M), and valine (V).

9. The recombinant protein of claim 8, wherein the hydrophobic, aliphatic amino acid is alanine (A).

10. The recombinant protein of any one of claims 1-9, wherein the composition binds and/or activates the granulocyte-macrophage colony stimulating factor receptor (GM-CSF-R-alpha or CSF2R).

11. The recombinant protein of claim 10, wherein the GM-CSF-R-alpha is expressed on the surface of a cell.

12. The recombinant protein of claim 11, wherein the cell is a hematopoietic progenitor cell.

13. The recombinant protein of claim 12, wherein the hematopoietic progenitor cell is an immune cell.

14. The recombinant protein of claim 12, wherein the hematopoietic progenitor cell is irradiated.

15. The recombinant protein of any one of the above claims, wherein the recombinant human GM-CSF is soluble.

16. The recombinant protein of any one of the above claims, wherein the recombinant human GM-CSF is functionally similar to sargramostim.

17. The recombinant protein of any one of the above claims, wherein the recombinant human GM-CSF comprises a plurality of molecular forms.

18. The recombinant protein of claim 17, wherein the molecular forms are selected from non-glycosylated, O-glycosylated, N-glycosylated and N+O glycosylated forms.

19. The recombinant protein of any one of the above claims, wherein the recombinant human GM-CSF is substantially free of hypermannosylated forms.

20. The recombinant protein of any one of the above claims, wherein the recombinant human GM-CSF resolves as three peaks when quantified by reversed phase high-performance liquid chromatography (RP-HPLC).

21. The recombinant protein of any one of the above claims, wherein the recombinant human GM-CSF provides no substantial peaks at a retention time of less than about 20 minutes when quantified by reversed phase high-performance liquid chromatography (RP-HPLC).

22. The recombinant protein of any one of the above claims, wherein the recombinant human GM-CSF is substantially free of hypermannosylated forms when purified without the use of organic solvents.

23. The recombinant protein of any one of the above claims, wherein the recombinant human GM-CSF is substantially free of hypermannosylated forms, optionally when purified without the use of a reversed phase C4 HPLC column.

24. A nucleic acid molecule encoding the recombinant human GM-CSF of any one of the above claims.

25. The nucleic acid of claim 24, wherein the nucleic acid molecule has a codon-optimized sequence.

26. A non-human host cell expressing the nucleic acid molecule of claim 23 or 24.

27. The host cell of claim 26, wherein the host cell is a yeast cell.

28. The host cell of claim 27, wherein the yeast cell is a non-methylotrophic yeast cell.

29. The host cell of claim 28, wherein the host cell is a Saccharomyces cerevisiae cell.

30. A pharmaceutical composition comprising a recombinant human GM-CSF of any one of the above claims and a pharmaceutically acceptable excipient or carrier.

31. A method of treating a patient or subject who is undertaking or has undertaken a cancer therapy, or who is undertaking or has undertaken a bone marrow transplant, and/or who had been acutely exposed to myelosuppressive doses of radiation; the method comprising administering to the patient a therapeutically effective amount of the pharmaceutical composition of claim 30.

32. The method of claim 31, wherein the patient is treated by modulating clonal expansion, survival, differentiation and activation state of hematopoietic progenitor cells.

33. The method of claim 31, wherein the patient is treated by modulating a myelomonocytic cell lineage, by promoting the proliferation of megakaryocytic and erythroid progenitors.

34. The method of claim 31, wherein the patient is treated by modulating hematopoietic progenitor cells, by stimulating the survival, proliferation and activation of neutrophils, macrophages and/or dendritic cells.

35. The method of claim 31, wherein the patient is treated following bone marrow transplant by modulating hematopoietic progenitor cells, by stimulating the survival, proliferation and activation of neutrophils, macrophages and/or dendritic cells.

36. A method of therapy, comprising
administering to a patient a therapeutically effective amount of the pharmaceutical composition of claim 30 or
contacting cells with an effective amount of the pharmaceutical composition of claim 30 and administering therapeutically effective amount of the cells,
wherein the therapy:
accelerates neutrophil recovery and/or to reduce the incidence of infections following induction chemotherapy;
mobilizes hematopoietic progenitor cells into peripheral blood for collection by leukapheresis and transplantation;
accelerates of myeloid reconstitution following autologous or allogeneic bone marrow or peripheral blood progenitor cell transplantation;
treats delayed neutrophil recovery or graft failure after autologous or allogeneic bone marrow transplantation; and/or
treats hematopoietic syndrome of acute radiation syndrome (H-ARS).

37. A method for treating an infection with a coronavirus, comprising: administering an effective amount of a composition comprising the recombinant human granulocyte macrophage-colony stimulating factor (GM-CSF) protein of any one of claims 1-22 to a patient in need thereof.

38. The method of claim 36, wherein the coronavirus is a betacoronavirus, optionally selected from severe acute respiratory syndrome coronavirus 2 (SARS-COV-2), SARS-CoV, Middle East respiratory syndrome-corona virus (MERS-COV), HCoV-HKU1, and HCoV-OC43.

39. The method of claim 37, wherein the coronavirus is an alphacoronavirus, optionally selected from HCoV-NL63 and HCoV-229E.

40. The method of claim 39, wherein the coronavirus is SARS-COV-2.

41. The method of claim 40, wherein the patient is afflicted with COVID-19.

42. The method of any one of claims 37-41, wherein the patient is afflicted with one or more of fever, cough, shortness of breath, diarrhea, upper respiratory symptoms, lower respiratory symptoms, pneumonia, and acute respiratory syndrome.

43. The method of any one of claims 37-42, wherein the patient is hypoxic.

44. The method of any one of claims 37-43, wherein the patient is afflicted with respiratory distress.

45. The method of any one of claims 37-44, wherein the method prevents or mitigates development of acute respiratory distress syndrome (ARDS) in the patient.

46. The method of any one of claims 37-45, wherein the method improves oxygenation in the patient.

47. The method of any one of claims 37-46, wherein the method prevents or mitigates a transition from respiratory distress to cytokine imbalance in the patient.

48. The method of any one of claims 37-47, wherein the method reverses or prevents a cytokine storm.

49. The method of claim 48, wherein the method reverses or prevents a cytokine storm in the lungs or systemically.

50. The method of claim 48 or 49, wherein the cytokine storm is selected from one or more of systemic inflammatory response syndrome, cytokine release syndrome, macrophage activation syndrome, and hemophagocytic lymphohistiocytosis.

51. The method of claim 48 or 49, wherein the method reverses or prevents excessive production of one or more inflammatory cytokines.

52. The method of claim 51, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

53. The method of any one of claims 37-52, wherein the method causes a decrease in viral load in the patient relative to before treatment.

54. A method for treating or preventing a viral infection in a subject in need thereof, comprising:
providing plasma from a donor subject who has recovered from the viral infection,
the plasma comprising IgG, IgM and/or IgA antibodies directed against the virus causing the infection and
the donor subject having been treated with the recombinant human granulocyte macrophage-colony stimulating factor (GM-CSF) protein of any one of claims 1-22 to stimulate production of the antibodies; and
administering the plasma to the subject in need thereof.

55. A method for treating or preventing a viral infection in a subject in need thereof, comprising:
administering the recombinant human granulocyte macrophage-colony stimulating factor (GM-CSF) protein of any one of claims 1-22 to a donor subject who has recovered from the viral infection;
isolating plasma from the donor subject, the plasma comprising IgG, IgM and/or IgA antibodies directed against the virus causing the infection; and
administering the plasma to the subject in need thereof.

56. The method of claim 54 or 55, wherein the method provides passive immunization against the virus to the subject in need thereof.

57. The method of any one of claims 54-56, wherein the IgG, IgM and/or IgA antibodies specifically bind to a viral antigen.

58. The method of claim 57, wherein the IgG, IgM and/or IgA antibodies neutralize the virus.

59. The method of claim 57 or 58, wherein the IgG, IgM and/or IgA antibodies prevent or diminish infection of a cell by the virus.

60. The method of any one of claims 54-59, wherein the viral infection is selected from a betacoronavirus infection, optionally selected from severe acute respiratory syndrome coronavirus 2 (SARS-COV-2), severe acute respiratory syndrome coronavirus (SARS-CoV-1), Middle East Respiratory Syndrome-Corona Virus (MERS-COV), HCoV-HKU1, and HCoV-OC43 infection.

61. The method of any one of claims 54-60, wherein the viral infection is selected from an alphacoronavirus infection, optionally selected from HCoV-NL63 and HCoV-229E infection.

62. The method of claim 61, wherein the betacoronavirus infection is severe acute respiratory syndrome (SARS).

63. The method of claim 61, wherein the betacoronavirus infection is, or is associated with, coronavirus disease 2019 (COVID-19).

64. The method of any one of claims 54-63, wherein the viral infection is an influenza infection, optionally selected from Type A, Type B, Type C, and Type D influenza virus infection.

65. The method of claim 64, wherein the influenza infection is pandemic 2009 influenza A (H1N1) or avian influenza A (H5N1).

66. The method of any one of claims 54-65, wherein the donor subject has tested positive for the viral infection prior to recovery.

67. The method of any one of claims 54-66, wherein the donor subject has resolution of viral infection symptoms prior to donation.

68. The method of any one of claims 54-67, wherein the donor subject has tested positive for antibodies directed against the virus using a serological test.

69. The method of any one of claims 54-68, wherein the donor subject demonstrates measurable neutralizing antibody titers.

70. The method of claim 69, wherein the neutralizing antibody titers are at least about 1:160.

71. The method of any one of claims 54-70, wherein the plasma is isolated from a blood sample from the donor subject.

72. The method of claim 71, wherein the plasma is isolated via plasmapheresis.

73. The method of any one of claims 54-72, wherein the plasma comprises a therapeutically effective amount of the IgG, IgM and/or IgA antibodies directed against the virus causing the infection.

74. A method of making a recombinant producing a composition comprising a recombinant human granulocyte macrophage-colony stimulating factor (GM-CSF) comprising:
(a) obtaining a cell transfected with a nucleic acid encoding a recombinant human granulocyte macrophage-colony stimulating factor (GM-CSF), comprising an amino acid sequence having at least about 97% identity with SEQ ID NO: 2 and having a substitution or deletion at position N37, G38 and/or T39 or a position corresponding thereto, or an extract thereof;
(b) purifying the GM-CSF from the transfected cell using one or more HPLC columns, wherein the purification is in the absence of an organic solvent; and
(c) collecting the purified GM-CSF, the purified GM-CSF being substantially free of hypermannosylated GM-CSF forms.

75. The method of claim 74, wherein the cell is a yeast cell.

76. The method of claim 75, wherein the yeast cell is Saccharomyces cerevisiae.

77. The method of claim 74, wherein the cell is a mammalian cell.

78. The method of claim 77, wherein the mammalian cell is a Chinese hamster ovary (CHO) cell.

79. The method of claim 78, wherein the transfection of a mammalian cell, such as a CHO cell, during production of the recombinant protein increases expression levels of the recombinant protein, as compared to a method of production using a non-mammalian cell.

80. The recombinant protein of claim 74, wherein the recombinant human GM-CSF shows enhanced function as compared to sargramostim.

81. The recombinant protein of claim 1 produced by transfection of a mammalian cell such as a CHO cell, wherein the recombinant human GM-CSF shows enhanced function as compared to sargramostim.

82. A composition comprising a recombinant human granulocyte macrophage-colony stimulating factor (GM-CSF) protein, comprising an amino acid sequence having at least about 97% identity with SEQ ID NO: 1 or SEQ ID NO: 2 and having a N37Q substitution, or a position corresponding thereto,
wherein the GM-CSF is isolatable without substantial hyperglycosylated species, without the need for organic solvent purification.


SEQUENCE LISTING (2 sequences)

SEQ ID NO: 1 - protein, 127 aa, Artificial Sequence [sequence omitted]

SEQ ID NO: 2 - protein, 127 aa, Artificial Sequence
  Synthetic Sequence
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE